(19) United States
(12) Patent Application Publication
(10) Pub. No.: US 2016/0151144 A1
(43) Pub. Date: Jun. 2, 2016

(54) VASCULAR FILTER STENT

(71) Applicant: ADIENT MEDICAL, INC., PEARLAND, TX (US)

(72) Inventor: Mitchell Donn Eggers, Pearland, TX (US)

(73) Assignee: Adient Medical, Inc.

(21) Appl. No.: 15/014,975

(22) Filed: Feb. 3, 2016

Related U.S. Application Data
(63) Continuation of application No. 13/096,049, filed on Apr. 28, 2011, now abandoned.

(51) Int. Cl. A61F 2/01

(52) U.S. Cl. CPC A61F 2/01; A61F 2002/011; A61F 2210/0004; A61F 2/82

(57) ABSTRACT

A vascular filter stent is disclosed for deployment within a vessel for filtering of body fluids. A preferred embodiment is the placement of such vascular filter stent within the inferior vena cava (IVC) to filter emboli for the prevention of pulmonary embolism. By incorporating a stent into the filter design, vessel patency and filter positioning is maintained, while minimizing endothelialization thereby obviating the long term complications of conventional metal VC filters such as filter migration and increased deep vein thrombosis.

### FIELD OF THE INVENTION

The present invention relates generally to a vascular filter and more particularly to a vascular filter stent deployed within a vessel for filtering of body fluids. A preferred embodiment is the placement of such vascular filter stent within the inferior vena cava (IVC) for the prevention of pulmonary embolism.

### BACKGROUND OF THE INVENTION

Between 100,000 to 300,000 Americans die annually from pulmonary embolism (PE)—more than breast cancer and AIDS combined—representing the 3rd leading cause of death in the US [1-5]. A similar incidence of PE is found in Europe with approximately 370,000 annual deaths [6]. Moreover, PE is the 3rd most common cause of death in trauma patients that survive the first 24 hours. An estimated 25% of all hospitalized patients have some form of deep vein thrombosis (DVT) which is often clinically unapparent unless PE develops [7]. On average, 33% of DVT will progress to symptomatic PE of which 10% will be fatal [6].

The US Surgeon General has recognized this alarming statistic and in 2008 issued a formal Call to Action to Prevent DVT and PE [1]. Unfortunately, DVT/PE disproportionately affects the elderly, in part due to prolonged periods of inactivity following medical treatment. The incidence is relatively low under the age of 50 (1/100,000), then accelerates exponentially reaching 1000/100,000 by the age of 85 [8]. Consequently the US Surgeon General has proclaimed that the growth in number of DVT/PE cases with an aging US population may outpace the population growth in the absence of better prevention [1].

Risk factors for PE arising from DVT follow Virchow's Triad [9]: (i) endothelial injury, (ii) hypercoaguability, and (iii) hemodynamic changes (stasis or turbulence). Hence specific risk factors include hip and knee arthroplasty, abdominal, pelvic and extremity surgeries, pelvic and long bone fractures, prolonged immobility such as prolonged hospital stays and air travel, paralysis, advanced age, prior DVT, cancer, obesity, COPD, diabetes and CHF. Orthopedic surgeons are especially concerned since their patients carry a 40%-80% risk for DVT and PE following knee and hip surgeries in the absence of prophylactic treatment [10-12].

The American Academy of Orthopaedic Surgeons (AAOS) has issued guidelines for PE prophylaxis. Basically, patients at standard risk should be considered for chemoprophylactic agents such as aspirin, low molecular weight heparin (LMWH), synthetic pentassaccharides, or warfarin, in addition to intra-operative and/or immediate postoperative mechanical prophylaxis [13].

Aspirin has a 29% relative risk reduction in symptomatic DVT and a 58% relative risk reduction in fatal PE [14]. LMWH carries a 30% risk reduction in DVT and has been proven more effective than unfractionated heparin in high risk groups such as hip and knee arthroplasty [7]. Warfarin started within 24 to 48 hours of initiating heparin with a goal of achieving international normalized ratio (INR) results between 2 and 3 as secondary thromboprophylaxis for 3 months reduces the risk of recurrent venous thromboembolism (VTE) by 90% as compared with placebo [15, 16]. Mechanical prophylaxis, consisting of pneumatic compression devices that repeatedly compress the legs with an air bladder, are also utilized in conjunction with anticoagulants to reduce the occurrence of PE.

The duration of prophylaxis depends on the source of potential DVT. Current recommendations for prophylaxis consist of a minimum 7 days and up to 30 days for many orthopedic surgeries. Specifically for orthopedic trauma, DVT prophylaxis is continued until patient mobilization (32%), inpatient discharge (19%), 3 weeks postop (16%), 6 weeks postop (27%), and in rare circumstances greater than 6 weeks (7%) [17]. Studies indicate that hypercoaguability persists for at least one month after injury in 80% of trauma patients [18]. Regarding total knee and hip arthroplasty and cancer surgeries, 35 day prophylactic treatment is recommended [12, 19]. Overall, prophylactic treatment for possible VTE is often warranted for up to 6 weeks following trauma or major surgery.

Contraindications for chemoprophylaxis include active bleeding, hemorrhagic diathesis, hemorrhagic stroke, neurologic surgery, excessive trauma, hemothorax, pelvic or lower extremity fractures with intracranial bleeding, anticoagulation interruption, and recent DVT/PE patients undergoing surgery.

For patients who are contraindicated for the above-mentioned anti-coagulation prophylaxis, or where anti-coagulation therapy has failed, the AAOS, American College of Physicians, and the British Committee of Standards in Haematology all recommend the use of inferior vena cava (IVC) filters [13, 20, 21]. These intravascular metal filters are deployed via catheter into the IVC to essentially catch emboli arising from DVT before reaching the lungs resulting in PE. Furthermore, the British Committee of Standards in Hematology recommends IVC filter placement in pregnant patients who have contraindications to anticoagulation and develop extensive VTE shortly before delivery (within 2 weeks).

The Eastern Association for Surgery of Trauma further recommends prophylactic IVC filters placed in trauma patients who are at increased risk of bleeding and prolonged immobilization [22]. Such prophylactic recommendation follows studies that demonstrate a low rate of PE in patients with severe polytrauma who underwent IVC placement [23-25]. In fact the fastest growing indication of overall IVC filter usage, from 49,000 in 1999 to 167,000 in 2007 with a projected 259,000 units for 2012, is the prophylactic market utilizing retrievable IVC filters [26, 27].

Example vascular filters primarily for IVC placement are disclosed in U.S. Pat. No. 4,425,908; U.S. Pat. No. 4,817,600; U.S. Pat. No. 5,626,605; U.S. Pat. No. 6,146,404; U.S. Pat. No. 6,217,600 B1; U.S. Pat. No. 6,258,026 B1; U.S. Pat. No. 6,497,709 B1; U.S. Pat. No. 6,506,205 B2; U.S. Pat. No. 6,517,559 B1; U.S. Pat. No. 6,620,183 B2; U.S. Pat. App. Pub. No. 2003/0176888; U.S. Pat. App. Pub. No. 2004/0193209; U.S. Pat. App. Pub. No. 2005/0267512; U.S. Pat. App. Pub. No. 2005/0267515; U.S. Pat. App. Pub. No. 2006/0206138 A1; U.S. Pat. App. Pub. No. 2009/0192543 A1; U.S. Pat. App. Pub. No. 2009/0299403 A1; U.S. Pat. App. Pub. No. 2010/0042135 A1; and U.S. Pat. App. Pub. No. 2010/0174310 A1.

IVC filter efficacy has been demonstrated in several class I and II evidence studies [22, 28-30]. Most of the earlier filters installed were expected to be permanent fixtures since endothelialization occurs within 7-10 days making most models impractical to remove without irreversible vascular damage leading to life threatening bleeding, dissection of the IVC, and thrombosis. Although these permanent filters have prevented PE, they have been shown to actually increase the risk of recurrent DVT over time.

Specifically, a Cochrane review [31] on the use of IVC filters for the prevention of PE cites a level I randomized prospective clinical trial by Decousus et al. [32] wherein the incidence of DVT with the IVC filter cohort increased almost 2-fold: (i) 21% incidence of recurrent DVT in the filter cohort vs. 12% in the non-filter LMWH cohort at 2 years (p=0.02), and (ii) 36% incidence of recurrent DVT in the filter cohort vs. 15% in the non-filter group at 8 years (p=0.042) [33]. However, the filters did reduce the occurrence of PE; the filter cohort experiencing only 1% PE vs. the non-filter cohort posting 5% PE in the first 12 days (p=0.03). No statistically significant difference in mortality rate was seen in any time frame investigated. Apparently the initial benefit of reduced PE with permanent IVC filters is offset by an increase in DVT, without any difference in mortality.

In addition to increased incidence of DVT for prolonged IVC filter deployment, filter occlusion has been reported with a 6% to 30% occurrence, as well as filter migration (3% to 69%), venous insufficiency (5% to 59%), and post thrombotic syndrome (13% to 41%) [34-36]. Complications from insertion including hematoma, infection, pneumothorax, vocal cord paralysis, stroke, air embolism, misplacement, tilting arteriovenous fistula, and inadvertent carotid artery puncture have an occurrence rate of 4%-11% [37].

Temporary or retrievable IVC filters have been marketed more recently intended to be removed once the risk of PE subsides, and hence circumvent many of the deleterious complications of permanent filters. The retrievable filters feature flexible hooks, collapsing components, and unrestrained legs to ease retrieval. Unfortunately these same features have led to unwanted filter migration, fatigue failure, IVC penetration, fragment migration to hepatic veins and pulmonary arteries, filter tilt, and metallic emboli [38-43]. Since 2005, 921 adverse filter events have been reported to the FDA including 328 device migrations, 146 device detachments (metallic emboli), 70 perforations of the IVC, and 56 filter fractures [44]. Some retrievable brands post alarming failure rates such as the Bard Recovery filter with 25% fracturing over 50 months which embolized end organs. 71% of the fractures embolized to the heart caused life threatening ventricular tachycardia, tamponade, and sudden death in some cases. An alternative retrievable model, Bard G2, resulted in 12% fractures over 24 months [45]. Such prevalence of device fractures is postulated to be directionally proportional to indwell time.

These failures and others prompted the FDA in August 2010 to issue a formal communication stating that “FDA recommends that implanting physicians and clinicians responsible for the ongoing care of patients with retrievable IVC filters consider removing the filter as soon as protection from PE is not longer needed” [44]. Even though these types of retrievable filters are intended to be removed in months time, several studies indicate that approximately 70%-81% of patients with retrievable IVC filters fail to return to the hospital for filter removal, thereby exposing hundreds of thousands of patients to the life-threatening adverse events of prolonged retrievable IVC filter placement [41, 44, 46-48]. These patients are either lost to follow-up, or refuse to have the filters removed in the absence of complications.

### BRIEF SUMMARY OF THE INVENTION

The present invention comprises systems and methods for filtering fluids. Certain embodiments comprise a novel vascular filter stent that prevents pulmonary embolism by capturing and restraining emboli within a body vessel. The vascular filter stent, according to certain aspects of the invention, possesses various advantages over all conventional vascular filters, including permanent, temporary, and optional IVC filters. Most importantly, the vascular filter stent disclosed herein is fabricated with a stent that serves as a circumferential mount for the capture elements in addition to providing vessel patency, and avoids endothelialization characteristic of metal filters with barbed struts. Hence the increased incidence of DVT observed with metal IVC filters due to inherent vessel damage from the metal struts is obviated. Moreover, the vascular filter elements are manufactured from collapsible materials which do not adversely impact end organs as exhibited by conventional metal IVC filters that migrate and often become fractionated. By incorporating a stent design with proven vessel retention, the vascular filter stent also obviates filter migration. Finally, the stent can be fabricated with a bioactive surface coating such as heparin to provide lasting anticoagulation.

### BRIEF DESCRIPTION OF THE DRAWINGS

FIG. 1a is a cut-away isometric view of one embodiment of the vascular filter stent that includes a plurality of capture elements attached to the stent for filtering substances such as emboli.

FIG. 1b features the capture elements of FIG. 1a in detail.

### DETAILED DESCRIPTION OF THE INVENTION

Embodiments of the present invention will now be described in detail with reference to the drawings, which are provided as illustrative examples so as to enable those skilled in the art to practice the invention. Notably, the figures and examples below are not meant to limit the scope of the present invention to a single embodiment, but other embodiments are possible by way of interchange of some or all of the described or illustrated elements. Wherever convenient, the same reference numbers will be used throughout the drawings to refer to same or like parts. Where certain elements of these embodiments can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the components referred to herein by way of illustration.

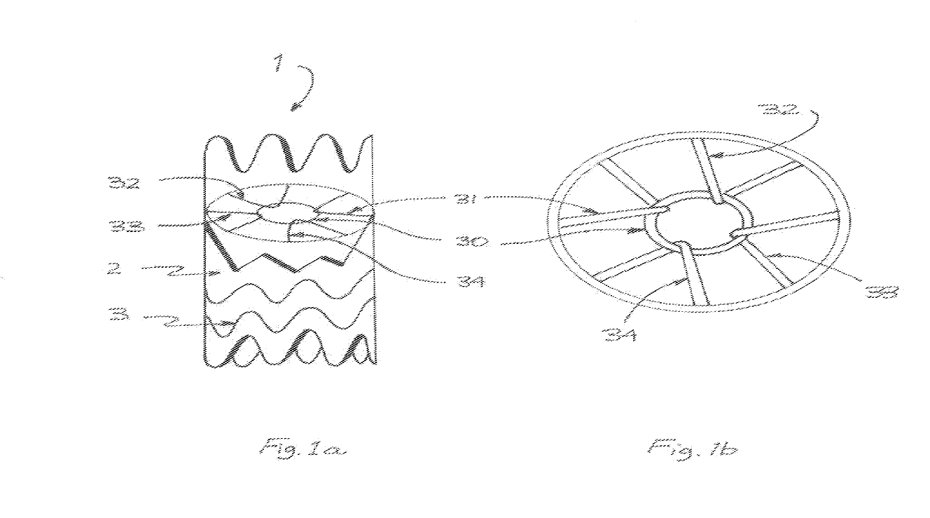

Referring to the embodiment depicted in FIGS. 1a, b, a vascular filter stent 1 consists of an outer, circumferential stent 2 for supporting a plurality of collapsible filter capture elements (30-34) and to maintain vessel patency. The capture elements are purposely designed to be collapsible for catheter-based installation and to avoid end organ damage. The supporting stent 2 is shown to be fabricated as an artificial vascular graft supported by undulating supporting structures 3.

Collapsible capture elements can be fabricated with numerous materials. Plausible materials include any suture such as surgical gut, Vicryl (polyglactin 910), Monocryl (poliglecaprone 25), PDS II (polydioxanone), silk, Ethilon (nylon), Nurolon (nylon), Mersilene (polyester fiber), Ethibond (polyester fiber), Prolene (polypropylene), Pronova Poly (hexafluoropropylene-VDF), Panacryl, Orthocord, Fiberwire, Novafil (polybutester), Vascufil (polybutester), Surgipro (polypropylene), Maxon (polytrimethylene carbonate), and Dexon.

As an alternative to assembling a plurality of capture elements, the vascular filter stent can be fabricated with composite mesh. Candidates for a mesh capture system include polypropylene such as C-QUR, polypropylene encapsulated by polydioxanone as in PROCEED, polypropylene co-knitted with polyglycolic acid fibers as in Bard Sepramesh IP Composite, polyethylene terephathalate as in Parietiex Composite, and ePTFE used in DUALAMESH.

The circumferential stent element 2 in FIG. 1 serves to support the capture elements of the vascular filter stent, in addition to maintaining vessel patency and maintaining stationary filter positioning within the vessel upon expansion. Numerous types of stents conventionally employed as thoracic endoprostheses can be utilized. Such stents would include Gore TAG, Medtronic Talent and Valiant Systems, and Cook Zenith TX2 System. In particular, the Gore TAG is comprised of an artificial vascular graft fabricated with a fluoropolymer (expanded polytetrafluoroethylene ePTFE and fluorinated ethylene propylene or FEP) combined with a Nitinol supporting structure. Alternatively, the stent component of the vascular filter stent can be fabricated with only the supporting structure (without the artificial vascular graft) utilizing Nitinol, Elgiloy, Phynox, 316 stainless steel, MP35N alloy, titanium alloy, platinum alloy, niobium alloys, cobalt alloys, and tantalum wire.

A preferred installation of the vascular filter stent is via intravenous insertion with a catheter requiring only a local anesthetic. Much like deployment of a conventional thoracic endoprosthesis or stent, vascular filter stent is collapsed and compressed within a delivery catheter. For IVC deployment, the delivery catheter is inserted into the patient's vasculature of convenient location, such as the femoral vein. Subsequently, the delivery catheter is fed through the vasculature until reaching the desired deployment location, typically just inferior to the renal veins. Next the compressed vascular filter stent is allowed to expand and subsequently the catheter housing is removed from the vein. Consequently as a thrombosis event releases an embolus, the embolus is captured by the vascular filter stent and is prevented from traveling to the heart and lungs thereby preventing a potentially fatal PE.

Although the present invention has been described with reference to specific exemplary embodiments, it will be evident to one of ordinary skill in the art that various modifications and changes may be made to these embodiments without departing from the broader spirit and scope of the invention. Accordingly, the specification and drawings are to be regarded in an illustrative rather than a restrictive sense.

### REFERENCES

[1] Goldhaber S Z, Ortel T L. The Surgeon General's Call to Action to Prevent Deep Vein Thrombosis and Pulmonary Embolism, Office of the Surgeon General (US), National Heart, Lung, and Blood Institute (US). Rockville (Md.). 2008
[2] Spencer F A, Emery C, Lessard D, Anderson F, Emani S, Aragam J et al. The Worcester Venous Thromboembolism study; a population-based study of the clinical epidemiology of venous thromboembolism. J Gen Intern Med 2006 Jul; 21 (7); 722-7.
[3] Bick R L. Hereditary and acquired thrombophilia: preface. Semin Thromb Hemost 1999; 25;251-3.
[4] Agudelo J F, Morgan S J, Smith W R. Venous Thromboembolism in Orthopedic Trauma Patients, Orthopedics. 2005 October;28(10):1164-71.
[5] Tapson V F. Acute pulmonary embolism. N Engl J Med, 2008, 358, 10. 1037-52.
[6] Goldhaber S Z, Visani L, De Rosa M. Acute PE: clinical outcomes in the International Cooperative PE Registry (ICOPER). Lancet 1999. 353. 1386-9.
[7] Geerts W H, Jay R M, Code Kl, et al. A comparison of low-dose heparin with low-molecular weight heparin as prophylaxis against venous thromboembolism after major trauma. N Engl J Med. 1996; 335:701-7.
[8] Silverstein D M, Heit J A, Mohr D N, Petterson T M, O'Fallon W M, Meltron L J, 3^{rd}. Trends int eh incidence of deep vein thrombosis and pulmonary embolism: a 25-year population-based study. Arch Intern Med 1998;158(6). 585-93.
[9] Von V R. Weitere untersuchungen ueber die verstopfung der lungenarterien and ihre folge. Traube's Beitraege exp Path u Physiol, 1846; 2:21-31.
[10] Goldhaber S Z, Savage D D, Garrison R J, et al. Risk factors for pulmonary embolism: The Framingham Study. Am J Med. 1983; 74: 1023-1028.
[11] Coon W W. Epidemiology of venous thromboembolism. Ann Surg. 1977; 186:149-164.
[12] Muntz J E, Michota F A. Prevention and management of venous thromboembolism in the surgeicl patient: options bye surgery type and individual patient risk factors, Am J of Surg, 2010; 199, S11-20.
[13] American Academy of Orthopaedic Surgeons Clinical Guideline on Prevention of Pulmonary Embolism in Patients Undergoing Total Hip or Knee Arthroplasty, American Academy of Orthopaedic Surgeons, May 2007.
[14] Pulmonary Embolism Prevention (PEP) Trial Collaborative Group: Prevention of pulmonary embolism and DVT with low dose aspirin: pulmonary embolism prevention (PEP) trial. Lancet. 2000; 355: 1295-1302
[15] Prins M H, Hutten B A, Koopman M M, et al. Longterm treatment of venous thromboembolic disease. Thromb Haemost 1999;82:892-8.
[16] Tran H, McRae S, Ginsberg J. Anticoagulant Treatment of Deep Vein Thrombosis and Pulmonary Embolism. Cardiology Clinics, 2008, 26, 235-50.
[17] Morgan S J, Jeray K J, Phieffer L S, Grisby J H, Bosse M J, Kellam J F. Attitudes of orthopaedic trauma surgeons regarding current controversies in the management of pelvic and acetabular fractures. J Orthop Trauma. 2001; 15:526-32.
[18] Meissner M H, Chandler W L, Elliot J S. Venous thromboembolism in trauma: a local manifestation of systemic hypercoagulability? J Trauma. 2003; 4:224-31.
[19] Geerts W H, Bergqvist D, Pineo G F, et al. Prevention of venous thromboembolism: American College of Chest Physicians evidence-based clinical practice guidelines (8^{th} edition). Chest. 2008;133:3815-4535.
[20] Huo M H, Spyropoulos A C. The eighth American college of chest physicians guidelines on venous thromboembolism prevention: implications for hospital prophylaxis strategies. J Thromb Thrombolysis. 2011, February;31(2): 196-208.
[21] Baglin T P, Brush J, Streiff M. Guidelines on the use of vena cava filters. British Committee for Standard in Haematology, British J of Haematology, 2006, 134, 590-5.
[22] Rogers F B, Cipolle M D, Velmahos G, et al. Practice management guidelines for the prevention of venous thromboembolism in trauma patients: the East practice management guidelines work group. J Trauma. 2002; 53:142-164.
[23] Rosenthal D, Wellons E D, Lai K M, et al. Retrievable inferior venal cava filters: initial clinical results. Ann Vasc Surg 2006; 20:157-165.
[24] Gosin I S, Graham A M, Ciocca R G, et al. Efficacy of prophylactic vena cava filters in high risk trauma patients. Ann Vasc Surg 1997; 11:100-05.
[25] Spain D A, Richardson J D, Polk H C, et al. Routine prophylactic vena cava filter insertion in severely injured patients decreases the incidence of pulmonary embolism. J Am Coll Surg 1995; 180:641-47.
[26] Stein P D, Kayali F, Olson R E. Twenty-one year trends in the use of inferior vena cava filters. Arch Intern Med 2004; 164:1541-5.
[27] Kaufman J A, Kinney T B, Streiff M D, et al. Guidelines for the use of retrievable and convertible vena cava filters: report from the Society of Interventional Radiology multidisciplinary consensus conference. J Vasc Interv Radiol. 2006;17:449-459.
[28] Rodriquez J L, Lopez J M, Proctor M C, et al. Early placemen of prophylactic vena cava filters in injured patients at high risk for pulmonary embolism. J Trauma. 1996; 40:797-804.
[29] Langan E M III, Miller R S, Caset W J III, Carsten C G III, Graham R M, Taylor S M. Prophylactic inferior vena cava filters in trauma patients at high risk: follow-up examination and risk/benefit assessment. J Vasc Surg. 1999; 30:484-90.
[30] Greenfield L J, Proctor M C, Rodriquez J L, Luchette F A, Cipolle M D, Cho J. Posttrauma thromboembolism prophylaxis. J Trauma. 1997,42:100-03.
[31] Young T, Tang H, Hughes R. Vena cava filters for the prevention of pulmonary embolism (Review). The Cochrane Library 2010, Issue 2.
[32] Decousus H, Leizorovics A, Parent F, et al. A clinical trial of vena caval filters in the prevention of pulmonary embolism in patients with proximal deep-vein thrombosis, N England J Med. 338,7:409-15.
[33] The PREPIC Study Group. Eight-year follow-up of patients with permanent vena cava filters in the prevention of pulmonary embolism: the PREPIC randomized study. Circulation 2005; 112:416-22.
[34] Ray C E, Kaufman J A. Complications of vena cava filters. Abdom Imaging 1996; 21:368-74.
[35] Ballew K A, Philbrick J T, Becker D M. Vena cava filter devices. Clin Chest Med 1995; 16:295-305.
[36] Streiff M B. Vena cava filters: a comprehensive review. Blood 2000; 95:3669-77.
[37] Pons M, Riglietti A, van den Berg J C. The role of vena cava filters in the management of venous thromboembolism. J Cardiovasc Surg 2010;51: 355-64.
[38] Usoh F, Hignorani A, Ascher E, et al. Long-term follow-up for surperior vena cava filter placement. Ann Vasc Surg. 2009;23:350-4.
[39] Turba U C, Arsian B, Meuse M, et al. Gunther Tulip filter retrieval experience: predictors of successful retrieval. Cardiovasc Intervent Radiol. 2009
[40] Kinney T B. Update on inferior vena cava filters. J Vasc Interv Radiol. 2003;14:425-40.
[41] Grande W J, Trerotola S O, Reilly P M, et al. Experience with the recovery filter as a retrievable inferior vena cava filter. J Vasc Interv Radiol 2005; 16:1189-93.
[42] Kirilcuk N N, Herget E J, Dicker R A, et al. Are temporary inferior vena cava filters really temporary? Am J Surg 2005; 190:858-63.
[43] Kumar B C, Chakraverty Z, Zealley I. Failed retrieval of potentially retrievable IVC filters: a report of two cases. Cardiovasc Intervent Radiol 2006; 29: 126-7.
[44] Removing Retrievable Inferior Vena Cava Filters: Initial Communication. FDA Division of Small Manufacturers, International and Consumer Assistance, Aug. 9, 2010.
[45] Nicholson W, Nicholson W J, Tolerico P, et al. Prevalence of fracture and fragment embolization of Bard retrievable vena cava filters and clinical implications including cardiac perforation and tamponade. Arch Intern Med. 2010.
[46] Karmy-Jones R, Jurkovich G H, Velmahos G C, et al. Practice patterns and outcomes of retrievable vena cava filters in trauma patients: an AAST multicenter study. J Trauma. 2007; 62: 17-25.
[47] Tschoe M, Kim H S, Brotman D J, et al. Retrievable vena cava filters: a clinical review. J Hosp Med 2009, 4;7: 441-8.
[48] Dabbagh O, Nagam N, Chitima-Matsiga R, et al. Retrievable inferior vena cava filters are not getting retrieved Where is the gap? Thrombosis Rsch 2010. 126: 493-7.

1.-18. (canceled)

19. A vascular filter stent fabricated for inferior vena cava (IVC) deployment comprising:
a circumferential element stent, wherein the circumferential element stent is absorbable and fabricated with undulating support structures effective to maintain IVC patency after deployment; and
a plurality of collapsible capture elements affixed to the absorbable circumferential element stent for capturing or retarding substances flowing in the IVC, wherein the collapsible capture elements are fabricated from an absorbable material.

20. The vascular filter stent set forth in claim 1, wherein more than one capture element has both ends attached to the absorbable circumferential element stent to form a loop, such that collectively the loops form a capture basket.

21. The vascular filter stent set forth in claim 1, wherein more than one capture element has both ends attached to the absorbable circumferential element stent to form a loop, and at least one capture element serves to integrate the loops to form a capture basket.

22. The vascular filter stent as set forth in claim 1, wherein more than one capture element has both ends attached to the absorbable circumferential element stent to form a loop that does not extend to the radial center of the absorbable circumferential element stent, and at least one capture element serves to integrate the loops to form a capture basket.

23. The vascular filter stent set forth in claim 1, wherein the capture elements degrade in time.

24. The vascular filter stent set forth in claim 1, wherein the capture elements are absorbable sutures.

25. The vascular filter stent set forth in claim 1, wherein the absorbable circumferential element stent is fabricated of polydioxanone, polytrimethylene carbonate, polyglactin, polyglycolic acid, poliglecaprone, polyglytone, or polylacticoglycolic acid.

26. The vascular filter stent set forth in claim 1, wherein the absorbable circumferential element stent contains a bioactive surface for anti-coagulation.

27. An absorbable filter fabricated for inferior vena cava (IVC) deployment comprising:
a circumferential element stent, wherein the circumferential element stent is absorbable and fabricated with undulating support structures effective to maintain IVC patency after deployment; and
a capture basket affixed to the absorbable circumferential element stent for capturing or retarding substances flowing in the IVC.

28. The absorbable filter as set forth in claim 15, wherein the capture basket is a mesh.

29. The absorbable filter as set forth in claim 15, wherein the capture basket is fabricated from polyglycolic acid fibers, PROCEED, Bard Sepramesh IP Composite, or Parietiex Composite.

30. A method for delivering a vascular filter stent as claimed in 1 or an absorbable filter as claimed in claim 15 with a delivery catheter, wherein the delivery comprises:
inserting the vascular filter stent or absorbable filter, in compressed form, within a delivery catheter to a desired position within a vessel; and
deploying the vascular filter stent or absorbable filter in expanded form at the desired position within a vessel; and subsequently removing delivery catheter from the vessel.

31. The vascular filter stent set forth in claim 1, wherein the capture element absorbable material comprises polydioxanone, polytrimethylene carbonate, polyglactin, polyglycolic acid, poliglecaprone, polyglytone, or polylacticoglycolic acid.

32. The vascular filter stent set forth in claim 7, wherein the absorbable sutures, comprise Vicryl, Monocryl, PDS, PDS II, Dexon, Dexon II, Maxon, PLGA, Surgical Gut, Ethibond, Panacryl, or Caprosyn.
